(19) United States
(12) Patent Application Publication
(10) Pub. No.: US 2002/0119201 A1
(43) Pub. Date: Aug. 29, 2002

(54) Lubricated granules

(75) Inventor: Ole Simonsen, Soborg (DK)

Correspondence Address: NOVOZYMES NORTH AMERICA, INC., C/O NOVO NORDISK OF NORTH AMERICA, INC., 405 LEXINGTON AVENUE, SUITE 6400, NEW YORK, NY, 10174, US

(73) Assignee: Novozymes A/S, Bagsvaerd (DK)

(21) Appl. No.: 09/948,266

(22) Filed: Sep. 7, 2001

Related U.S. Application Data
(60) Provisional application No. 60/232,087, filed on Sep. 12, 2000.

(30) Foreign Application Priority Data
Sep. 8, 2000 (DK) PA 2000 01340

(51) Int. Cl. A61K 9/16; C11D 1/00

(52) U.S. Cl. 424/490; 510/349

(57) ABSTRACT

An active-containing granule is provided which comprises a lubricant on the outer surface of the granule, wherein the lubricant is in liquid form at 25° C. and has a viscosity of less than 5000 centipoise at 25° C.

### CROSS-REFERENCE TO RELATED APPLICATIONS

This application claims, under 35 U.S.C. 119, the benefit of Danish application no. PA 2000 01340, filed Sep. 8, 2000, and U.S. provisional application No. 60/232,087, filed Sep. 12, 2000, the contents of which are fully incorporated herein by reference.

### FIELD OF THE INVENTION

The present invention relates to a granule comprising an active component and an outer lubrication layer which inhibits formation of dusting of active component from the granule.

### BACKGROUND OF THE INVENTION

Granules containing active components, such as enzymes have been used for decades in various industries. Several technologies have been developed to provide such granules. One object of providing actives in the form of granules is to prevent or inhibit formation of active, because actives may cause health damage to persons handling the actives. Conventionally this has been achieved by providing active-containing granules comprising an active-containing core and various coating layers. An active-containing granule typically consist of a core and a coating, where an important feature of the coating is to reduce the formation of dust when handling the granulate.

WO 96/16151 relates to enzyme granules coated with a non aqueous liquid or a unctuous mixture and an anti-caking agent

WO 01/04279 and WO 00/01793 (unpublished at the date of priority) also disclose granules comprising a lubricant.

### SUMMARY OF THE INVENTION

We have developed a granule which in addition to coating layers also comprise a lubrication layer on the outer surface of the granule.

Accordingly the present invention relates to an active-containing granule comprising a lubricant on the outer surface of the granule, characterized by

(a) the lubricant is in liquid form at 25° C. and has a viscosity of less than 5000 centipoise at 25° C. or

(b) the lubricated granules have a relative friction coefficient which is less than 80% when compared to unlubricated granules when measured by a rheometer by using a tip speed of 50 rpm, a helix angle of 30 (compaction mode), using the 46 mm rotor and weighing 170 g granulate into a 50 mm testing container or

(c) the lubricant is a mineral oil or a combination thereof

Further aspects the invention relates to a process for producing a granule of the invention, compositions comprising the granule of the invention and to use of the granule of the invention.

### DETAILED DESCRIPTION OF THE INVENTION

Granules containing actives, such as enzymes, are dry particles which upon handling, are subjected to wear and tear (collision and friction) because individual granules collide with each other and cause “grinding” of the granule surface. This is believed to cause breakage of coating or even disintegration of the granule, where upon dry active, e.g. enzyme protein, may be liberated and form health damaging dust. However limiting these handling damages e.g. by lowering movements of individual granules relative to each other is undesirable because the granules should also possess good flow and mixing ability to ease the handling of the granules, which requires relative free movement of individual granules.

Without being bound to the this theory we believe that applying a lubricant on the outer surface will “grease” the individual granules, i.e. reduce the friction between granules, so as to provide good flow and mixing capabilities of the granule, while at the same time lowering frictional forces causing breakage of granules and release of active dust. Further the lubricant may also reduce the tackiness of eg. binders in a coating on which the lubricant is applied further preventing agglomeration of granules. Thus, lubricants as herein defined can serve as anti-agglomeration agents and wetting agents as well as reducing dust formation and granule breakage.

Besides for the lubricant, the active-containing granule of the invention suitably comprises an active-containing core and one or more coating layers as known in the art.

Definitions

The term lubricant is to be understood as any non-aqueous compound or mixture of compounds, which forms a liquid at 25° C. and atmospheric pressure. Preferably it has a viscosity of less than 5000 centipoise at these conditions.

The Core

The core contains the active(s), preferably enzyme(s). Besides of the actives(s) the core may be constructed in any way or of any material which provides the desired functional properties of the core material, e.g. the core may consist of materials which allows readily release of the actives(s) upon introduction to an aqueous medium.

Preferred constructions of the core includes:

Spray dried core particles, prepared by spray drying a liquid enzyme containing solution whereby small droplets of active-containing solution dry up to form an active-containing Is particulate material. Very small particles can be produced this way (Michael S. Showell (editor); Powdered detergents; Surfactant Science Series; 1998; vol. 71; page 140-142; Marcel Dekker).

Layered core particles, wherein an active is coated as a layer around a preformed core particle or adsorbed into the surface of the core particle. Particles of a desired size can be obtained this way if a useful core particle of the desired size can be found. This type of product is described in e.g. WO 97/23606 and WO 97/39116 incorporated by reference.

Extruded or pelletized core particles, wherein an active-containing paste is pressed to pellets or under pressure is extruded through a small opening and cut into particles which is subsequently dried. Such particles usually have a considerable size because of the material in which the extrusion opening is made (usually a plate with bore holes) sets a limit on the allowable pressure drop over the extrusion opening. Also very high extrusion pressures when using a small opening increases heat generation in the active-containing paste which may be harmful to the active. (Michael S. Showell (editor); Powdered detergents; Surfactant Science Series; 1998; vol. 71; page 140-142; Marcel Dekker) Prilled core particles, wherein an active-containing powder is immobilised in a solidified wax matrix. Prilled particles are usually prepared by suspending active powder in molten wax and spraying the suspension, e.g. through a rotating disk atomizer, into a cooling chamber where the droplets quickly solidify (Michael S. Showell (editor); Powdered detergents; Surfactant Science Series; 1998; vol. 71; page 140-142; Marcel Dekker).

Mixer granulation particles prepared by adding active, liquid and a dry powder composition of granulating components. The liquid and the powder in a suitable proportion is mixed and as the moisture of the liquid is absorbed in the dry powder, the components of the dry powder will start to adhere and agglomerate and particles will build up forming granules comprising the active. Such a process is described in 4,106,991 (NOVO NORDISK) and related documents EP 170360 B1 (NOVO NORDISK), EP 304332 B1 (NOVO NORDISK), EP 304331 (NOVO NORDISK), WO 90/09440 (NOVO NORDISK) and WO 90/09428 (NOVO NORDISK), all incorporated by reference.

The components of the core may be those known to the art including but not limited to:

Actives

The active may be any active component, which use benefits from being formulated into a granule. Such actives may be a pharmaceutical

The term “active” is meant to encompass all components, which when released from the granule in application of the granule in ma process, serves a purpose of improving the process. Suitable actives are those which are either subjects of deactivation and/or causing deactivation to other components in the a composition comprising the granule.

The active may be inorganic of nature, such as bleach components, or organic. Preferred actives are biologically active materials, such as catalytically active materials such as enzymes, pharmaceutical materials active in the human or animal body or agricultural chemicals such as herbicides, pesticides, bactericides and/or fungicides. Such compounds are usually very sensitive to the surrounding environment and may suitably be obtained from chemical processes or from fermenting microorganisms. Most preferred actives are peptides or polypetides such as enzymes.

An enzyme in the context of the present invention may be any enzyme or combination of different enzymes, which benefits from being granulated in order to be applicable for a specific use. Accordingly, when reference is made to “an enzyme” this will in general be understood to include combinations of one or more enzymes.

It is to be understood that enzyme variants (produced, for example, by recombinant techniques) are included within the meaning of the term “enzyme”. Examples of such enzyme variants are disclosed, e.g., in EP 251,446 (Genencor), WO 91/00345 (Novo Nordisk), EP 525,610 (Solvay) and WO 94/02618 (Gist-Brocades NV).

The enzyme classification employed in the present specification with claims is in accordance with Recommendations (1992) of the Nomenclature Committee of the International Union of Biochemistry and Molecular Biology, Academic Press, Inc., 1992.

Accordingly the types of enzymes which may appropriately be incorporated in granules of the invention include oxidoreductases (EC 1.-.-.-), transferases (EC 2.-.-.-), hydrolases (EC 3.-.-.-), lyases (EC 4.-.-.-), isomerases (EC 5.-.-.-) and ligases (EC 6.-.-.-).

Preferred oxidoreductases in the context of the invention are peroxidases (EC 1.11.1), laccases (EC 1.10.3.2) and glucose oxidases (EC 1.1.3.4), while preferred transferases are transferases in any of the following sub-classes:

a) Transferases transferring one-carbon groups (EC 2.1);

b) transferases transferring aldehyde or ketone residues (EC 2.2); acyltransferases (EC 2.3);

c) glycosyltransferases (EC 2.4);

d) transferases transferring alkyl or aryl groups, other that methyl groups (EC 2.5); and

e) transferases transferring nitrogeneous groups (EC 2.6).

A most preferred type of transferase in the context of the invention is a transglutaminase (protein-glutamine γ-glutamyltransferase; EC 2.3.2.13).

Further examples of suitable transglutaminases are described in WO 96/06931 (Novo Nordisk A/S).

Preferred hydrolases in the context of the invention are: Carboxylic ester hydrolases (EC 3.1.1.-) such as lipases (EC 3.1.1.3); phytases (EC 3.1.3.-), e.g. 3-phytases (EC 3.1.3.8) and 6-phytases (EC 3.1.3.26); glycosidases (EC 3.2, which fall within a group denoted herein as “carbohydrases”), such as α-amylases (EC 3.2.1.1); peptidases (EC 3.4, also known as proteases); and other carbonyl hydrolases].

In the present context, the term “carbohydrase” is used to denote not only enzymes capable of breaking down carbohydrate chains (e.g. starches) of especially five- and six-membered ring structures (i.e. glycosidases, EC 3.2), but also enzymes capable of isomerizing carbohydrates, e.g. six-membered ring structures such as D-glucose to five-membered ring structures such as D-fructose.

Carbohydrases of relevance include the following (EC numbers in parentheses):

α-amylases (3.2.1.1), β-amylases (3.2.1.2), glucan 1,4-α-glucosidases (3.2.1.3), cellulases (3.2.1.4), endo-1,3(4)-β-glucanases (3.2.1.6), endo-1,4-β-xylanases (3.2.1.8), dextranases (3.2.1.11), chitinases (3.2.1.14), polygalacturonases (3.2.1.15), lysozymes (3.2.1.17), β-glucosidases (3.2.1.21), α-galactosidases (3.2.1.22), β-galactosidases (3.2.1.23), amylo-1,6-glucosidases (3.2.1.33), xylan 1,4-β-xylosidases (3.2.1.37), glucan endo-1,3-β-D-glucosidases (3.2.1.39), α-dextrin endo-1,6-α-glucosidases (3.2.1.41), sucrose α-glucosidases (3.2.1.48), glucan endo-1,3-α-glucosidases (3.2.1.59), glucan 1,4-β-glucosidases (3.2.1.74), glucan endo-1,6-β-glucosidases (3.2.1.75), arabinan endo-1,5-α-L-arabinosidases (3.2.1.99), lactases (3.2.1.108), chitosanases (3.2.1.132) and xylose isomerases (5.3.1.5).

Examples of commercially available oxidoreductases (EC 1.-.-.-) include Gluzyme™ (enzyme available from Novo Nordisk A/S).

Examples of commercially available proteases (peptidases) include Kannase™, Everlase™, Esperase™, Alcalase™, Neutrase™, Durazym™, Savinase™, Pyrase™, Pancreatic Trypsin NOVO (PTN), Bio-Feed™ Pro and Clear-Lens™ Pro (all available from Novo Nordisk A/S, Bagsvaerd, Denmark).

Other commercially available proteases include Maxatase™, Maxacal™, Maxapem™, Opticlean™ and Purafect™ (available from Genencor International Inc. or Gist-Brocades).

Examples of commercially available lipases include Lipoprime™ Lipolase™, Lipolase™ Ultra, Lipozyme™, Palatase™, Novozym™ 435 and Lecitase™ (all available from Novo Nordisk A/S).

Other commercially available lipases include Lumafast™(Pseudomonas mendocina lipase from Genencor International Inc.); Lipomax™ (Ps. pseudoalcaligenes lipase from Gist-Brocades/Genencor Int. Inc.; and Bacillus sp. lipase from Solvay enzymes.

Examples of commercially available carbohydrases include Alpha-Gal™, Bio-Feed™ Alpha, Bio-Feed™ Beta, Bio-Feed™ Plus, Bio-Feed™ Plus, Novozyme™ 188, Celluclast™, Cellusoft™, Ceremyl™, citrozym™, Denimax™, Dezyme™, Dextrozyme™, Finizym™, Fungamyl™, Gamanase™, Glucanex™, Lactozym™, Maltogenase™, Pentopan™, Pectinex™, Promozyme™, Pulpzyme™, Novamyl™, Termamyl™, AMG™ (Amyloglucosidase Novo), Maltogenase™, Sweetzyme™ and Aquazym™ (all available from Novo Nordisk A/S).

The amount of enzyme to be incorporated in a granule of the invention will depend on the intended use of the granulate. For many applications, the enzyme content will be as high as possible or practicable.

The content of enzyme (calculated as percent pure enzyme protein per granule weight) in a granule of the invention will typically be in the range of from about 0.5% to 50% by weight of the enzyme-containing granule, but higher amounts such as 50-90% w/w are also suitable.

When, for example, a protease (peptidase) is incorporated in granules according to the invention, the enzyme activity (proteolytic activity) of the finished granules will typically be in the range of 1-20 KNPU/g. This unit for protease activity is Kilo Novo Protease Units per gram of sample (KNPU/g). The activity is determined relatively to an enzyme standard of known activity in KNPU/g. The enzyme standard is standardized by measuring for a given amount of enzyme the formation rate (μmol/minute) of free amino groups liberated from digestion of di-methyl-casein (DMC) in solution by the enzyme. The formation rate is monitored by recording the linear development of absorbance at 420 nm of the simultaneous reaction between the formed free amino groups and added 2,4,6-tri-nitro-benzene-sulfonic acid (TNBS). The digestion of DMC and the color reaction is carried out at 50° C. in a pH 8.3 boric acid buffer with a 9 min. reaction time followed by a 3 min. measuring time. A folder AF 220/1 is available upon request to Novo Nordisk A/S, Denmark, which folder is hereby included by reference.

Likewise, in the case of, for example, α-amylases, an activity of 10-500 KNU/g will be typical. The activity is determined relatively to an enzyme standard of known activity in KNU/g. The enzyme standard is standardized by measuring for a given amount of enzyme the formation rate (μmol/minute) of 2-chlor-4-nitrophenol liberated from digestion of 2-chlor-4-nitrophenyl-b-D-maltoheptaosid substrate by the enzyme and auxiliary alfa- and beta-glucosidase enzymes in solution. Kits for performing α-amylase assays are commercially available. One description of an α-amylase assay may be found in the leaflet AF318/1-GB available upon request from Novo Nordisk A/S, Denmark. For eg lipases, an activity in the range of 50-400 KLU/g will normally be suitable.

Usually the enzyme will be applied to the granulation process as an enzyme containing liquid. The enzyme containing liquid may be applied as a purified product in which the enzyme is dissolved or dispersed as crystalline and/or amorphous protein in an aqueous liquid in the form of an enzyme concentrate or the enzyme containing liquid may be in the form of a fermentation broth. The water in the liquid may be used as a liquid agent for the granulation process (supra).

Preformed Core Particles

In the case the granule of the invention is prepared by applying a layer of active around a preformed core particle, the preformed core particles are preferably carbohydrate based core particles such as particles formed from plant flours or products (cassava, manioc, cereal flours, sugars, dextrins etc.) or salt based core particles made from salts such as, alkali or earth alkali metal salts of sulfate, sulfite, phosphate, phosphonate, nitrate, chloride or carbonate or salts of simple organic acids such as citrate, malonate or acetate are preferred.

Fillers

Suitable fillers are water soluble and/or insoluble inorganic salts such as finely ground alkali sulphate, alkali carbonate and/or alkali chloride), clays such as kaolin (e.g. Speswhite™, English China Clay), bentonites, talcs, zeolites, and/or silicates.

Binders

Suitable binders are binders with a high melting point or no melting point at all and of a non waxy nature e.g. polyvinyl pyrrolidon, dextrins, polyvinylalkohol, cellulose derivatives, for example hydroxypropyl cellulose, methyl cellulose or CMC. A suitable binder is a carbohydrate binder such as Glucidex 21D available from Roquette Freres, France.

Fiber Materials

Pure or impure cellulose in fibrous form can be sawdust, pure fibrous cellulose, cotton, or other forms of pure or impure fibrous cellulose. Also, filter aids based on fibrous cellulose can be used. Several brands of cellulose in fibrous form are on the market, e.g. CEPO and ARBOCELL. In a publication from Svenska Trämjolsfabrikerna AB, “Cepo Cellulose Powder” it is stated that for Cepo S/20 cellulose the approximate maximum fiber length is 500 μm, the approximate average fibre length is 160 μm, the approximate maximum fibre width is 50 μm and the approximate average fibre width is 30 μm. Also, it is stated that CEPO SS/200 cellulose has an approximate maximum fibre length of 150 μm, an approximate average fibre length of 50 μm, an approximate maximum fiber width of 45 μm and an approximate average fiber width of 25 μm. Cellulose fibers with these dimensions are very well suited for the purpose of the invention. The words “Cepo” and “Arbocel” are Trade marks. A preferred fibrous cellulose is Arbocel™ BFC200. Also synthetic fibres may be used as described in EP 304331 B1 and typical fibres may be made of polyethylene, polypropylene, polyester, especially nylon, polyvinylformat, poly(meth)acrylic compounds.

Liquid Agents

A liquid agent is used in conventional mixer granulation processes for enabling the build up or agglomeration of the conventional granulating component particles into granules. The liquid agent is water and/or a waxy substance. The liquid agent is always used in a liquid phase in the granulation process but may later on solidify; the waxy substance if present, therefore, is either dissolved or dispersed in the water or melted. By the term “waxy substance” as used herein is meant a substance which possesses all of the following characteristics 1) the melting point is between 30 and 100° C., preferably between 40 and 60° C., 2) the substance is of a tough and not brittle nature, and 3) the substance possesses a certain plasticity at room temperature. Both water and waxy substance are liquid agents, i.e. they are both active during the formation of the granules; the waxy substance stays as a constituent in the finished granules, whereas the majority of the water is removed during a drying step. Examples of waxy substances are polyglycols, fatty alcohols, ethoxylated fatty alcohols, mono-, di- and triglycerolesters of higher fatty, acids, e.g. glycerol monostearate, alkylarylethoxylates, and coconut monoethanolamide.

If a high amount of waxy substance is used, relatively little water should be added, and vice versa. Thus, the liquid agent can be either water alone, waxy substance alone or a mixture of water and waxy substance. When a mixture of water and waxy substance is used, the water and the waxy substance can be added in any sequence, e.g. first the water and then the waxy substance, or first the waxy substance and then the water or a solution or suspension of the waxy substance in the water. Also, when a mixture of water and waxy substance is used, the waxy substance can be soluble or insoluble (but dispersible) in water. If water is used a liquid agent it may not be a part of the finished mixer granule as usually most of the water is dried off off at a subsequent drying of the mixer granules.

Active-Stabilizing or Active-Protective Agents

For enzymes stabilizing or protective agents may fall into several categories: alkaline or neutral materials, reducing agents, antioxidants and/or salts of first transition series metal ions. Each of these may be used in conjunction with other protective agents of the same or different categories. Examples of alkaline protective agents are alkali metal silicates,—carbonates or bicarbonates which provide a chemical scavenging effect by actively neutralizing e.g. oxidants. Examples of reducing protective agents are salts of sulfite, thiosulfite or thiosulfate, while examples of antioxidants are methionine, butylated hydroxytoluene (BHT) or butylated hydroxyanisol (BHA). Most preferred agents are salts of thiosulfates, e.g. sodium thiosulfate. Also enzyme stabilizers may be borates, borax, formates, di- and tricarboxylic acids and reversible enzyme inhibitors such as organic compounds with sulfhydryl groups or alkylated or arylated boric acids.

Crosslinking Agents

Crosslinking agents may be active-compatible surfactants eg ethoxylated alcohols, especially ones with 10 to 80 ethoxy groups.

Further suspension agents, mediators (for boosting bleach action upon dissolution of the granule in e.g. a washing application) and/or solvents may be incorporated in the granule core.

Coatings

One or more coating may be applied to the granule of the invention to provide desired properties of the granule. e.g. protection of the active in the core. The components of a coating may suitably be components, supra, used in the core, preferably with the exception of enzymes.

Conventional coatings as known to the art may suitably be used such as the coatings described in WO 89/08694, WO 89/08695, 270 608 B1 and/or PA 1998 00876 (Danish priority application unpublished at the priority date of this invention). Other examples of conventional coating materials may be found in U.S. Pat. No. 4,106,991, EP 170360, EP 304332, EP 304331, EP 458849, EP 458845, WO 97/39116, WO 92/12645A, WO 89/08695, WO 89/08694, WO 87/07292, WO 91/06638, WO 92/13030, WO 93/07260, WO 93/07263, WO 96/38527, WO 96/16151, WO 97/23606, U.S. Pat. Nos. 5,324,649, 4,689,297, EP 206417, EP 193829, DE 4344215, DE 4322229 A, DD 263790, JP 61162185 A and/or JP 58179492.

In a particular embodiment the coating may comprise minor amounts of a protective agent capable of reacting with a component capable of inactivating (being hostile to) the active, said component entering the granule from a surrounding matrix, i.e. before the component come into contact and inactivate the active. The protective agent may thus e.g. be capable of neutralizing, reducing or otherwise reacting with the component rendering it harmless to the active. Typical components capable of inactivating the active are oxidants such as perborates, percarbonates, organic peracids and the like.

Protective agents may fall into several categories: alkaline or neutral materials, reducing agents, antioxidants and/or salts of first transition series metal ions. Each of these may be used in conjunction with other protective agents of the same or different categories. Examples of alkaline protective agents are alkali metal silicates,—carbonates or bicarbonates which provide a chemical scavenging effect by actively neutralizing e.g. oxidants. Examples of reducing protective agents are salts of sulfite, thiosulfite or thiosulfate, while examples of antioxidants are methionine, butylated hydroxytoluene (BHT) or butylated hydroxyanisol (BHA). Most preferred agents are salts of thiosulfates, e.g. sodium thiosulfate. The amounts of protective agent in the coating may be 5-40% w/w of the coating, preferably 5-30%, e.g. 10-20%.

The coating should encapsulate the active-containing granule by forming a substantially continuous homogenous layer.

The coating may perform any of a number of functions in the granule, depending on the intended use of the granule. Thus, for example, a coating may achieve one or more of the following effects:

(i) some reduction of the active dust formation of an active-containing granule;

(ii) protection of actives(s) in the active-containing granule against oxidation by bleaching substances/systems (e.g. perborates, percarbonates, organic peracids and the like);

(iii) dissolution at a desired rate upon introduction of the granule into a liquid medium (such as an aqueous medium);

(iv) provide a better physical strength of the granule.

The coating may further comprise one or more of the following: anti-oxidants, chlorine scavengers, plasticizers, pigments, additional enzymes and fragrances.

Plasticizers useful in coating layers in the context of the present invention include, for example: polyols such as sugars, sugar alcohols, or polyethylene glycols (PEGs) having a molecular weight less than 1000; urea, phthalate esters such as dibutyl or dimethyl phthalate; and water.

Suitable pigments include, but are not limited to, finely divided whiteners, such as titanium dioxide or kaolin, coloured pigments, water soluble colorants, as well as combinations of one or more pigments and water soluble colorants.

In a preferred embodiment of the invention the granule of the invention is coated with a protective coating having a high constant humidity such as described in the Danish patent application WO 00/01793 pages 5-9 and examples hereby incorporated by reference.

The core and coating constituting the active-containing granulate to be lubricated are suitably spherical or near spherical and may suitable have an average diameter in their longest dimension in the range of 50-2000 μm, preferably 200-1200 μm, more preferably 400-800 μm or 50-200 μm.

The Lubricant

The lubricant of the invention is a compound or a mixture of compounds forming a non-aqueous liquid at 25° C. and which preferably has a viscosity of less than 5000 centipoises at 25° C., such as 500-5000 cP, preferably less than 4000 centipoises such as 500-4 000 cP, more preferably less than 3000 centipoises such as 500-3000 cP and most preferably less than 2500 centipoises such as 500-2500 cP. One advantage of using lubricants having a low viscosity is that they are considerably easier to apply as a thin layer in small amounts on a granule and that a homogenous distribution of the small amounts of lubricant on the entire granules surface of a granules is facilitated by a low viscosity. When using lubricants having a higher viscosity the lubricant tends to adhere inhomogenously to the granule surface in the form of sticky lumps. Another advantage of low viscosity lubricants is that especially low viscosity lubricants reduces the friction of granules when the lubricant is applied to the outer surface of said granules. Reduction of granule friction may suitable be measured by a rheometer. Hence, one aspect of the invention, as mentioned supra, relates to active-containing granules comprising a lubricant on the outer surface of the granule, wherein the lubricated granules have a relative friction coefficient which is less than 80%, e.g. 5-80%, when compared to unlubricated granules when measured by a rheometer by using a tip speed of 50 rpm, a helix angle of 3° (compaction mode), using the 46 mm rotor and weighing 170 g granulate into a 50 mm testing container. Preferably, the relative friction coefficient is less than 78%, e.g. 5-78%, more preferably less than 75%, e.g. 5-75%, most preferably less than 70%, e.g. 5-70%, compared to unlubricated granules.

The lubricant is preferably an organic compound or a mixture of organic compounds that satisfy the low viscosity requirements. Preferred lubricants are nonionic surfactants such as Softanol (e.g. Softanol 50) and/or Dobanol, natural refined mineral oils such as Whiteway T15 (an alkane oil), synthetic mineral oils, such as silicone oils, animal oils, plant oil or any suitable mixture.

Particular advantages are achieved when using a lubricant E which as a further feature are substantially not soluble in the granule material on which it is applied. One advantage of this embodiment is that the lubricant will not dissolve in and/or mix with the granule surface on which it is applied and disappear, e.g. by absorption, from the granule surface where it serves it function. Another advantage of this embodiment is that the lubricant and the coating material may be mixed prior to the application of lubricant and coating to the granules. This mixture or dispersion may thus be applied simultaneously to the granule and because of the insolubility of the lubricant in the coating material the lubricant may separate from the coating material to form an outer lubrication layer. The term “substantially not soluble” in this context means that less than 1 g lubricant can be dissolved in 1 kg of the granule material on which it is applied. The granule material, such as a coating layer on which the lubricant is applied is usually a hydrophilic water soluble material. Thus in these embodiments, preferred lubricants are hydrophobic. Especially, mineral oil lubricants are suitable and hence in one aspect the invention relates to active-containing granules comprising a mineral oil lubricant on the outer surface of the granules. Most preferred lubricants are mineral oil lubricants having a viscosity of less than 5000 centipoises which reduces the relative friction coefficient of lubricated granules to less than 80% when compared to unlubricated granules.

While lubrication of active-containing granules will grease and reduce friction between granules, rendering them less dusting and lowering the risk of breakage, we have also found that in some instances the greasing properties of the lubricant, also may cause the dry granules to become sticky and agglomerate if too much lubricant is applied. Agglomeration may be inhibited by powdering the granule with a dry particulate material, such as TiO₂, such as described in U.S. Pat. No. 4,106,991 example 22. However, this will counter act or even cancel the lubrication effect and we have found that lowering the amount of lubricant preferably to less than 1% w/w of the lubricated granule will prevent significant agglomeration, while maintaining the beneficiary greasing effect. Accordingly, to prevent agglomeration and/or stickiness of the granules to become a problem in handling active-containing granules and to avoid the cost and problems of adding various anti-agglomeration agents, addition the lubricant should preferably be applied in a very thin layer constituting less than 1% of the granule by weight, such as between 0.01% to 1% preferably less than 0.75% w/w, such as between 0.1% to 0.75%, more preferably about 0.5% w/w such as between 0.1% to 0.5% of the granule. Applying a very thin lubrication layer will be facilitated by using a low viscosity lubricant and/or a lubricant which is substantially not soluble in the granule material on which it is applied as described supra.

As stated, due to the nature and preferred low amounts of the lubricant on granules of the invention the use of an anti-caking agent to prevent agglomeration during manufacture or storage of granules may successfully be omitted. Hence in a preferred embodiment of the invention the granules of the invention are free of anti-caking agents applied on the lubrication layer.

Process for Preparing a Lubricated Granule

The lubricant may suitably be applied to an enzyme granule by adding the lubricant to the granule in suitable amounts and mixing this composition until the lubricant deposited and distributed on the surface of the granules. The mixing may be achieved by spraying the lubricant onto the enzyme granule in a high speed mixer or in a fluid bed coater. As said, in a preferred embodiment the process of preparing granules of the invention is conveniently free of any steps adding anti-agglomeration agents onto the lubrication layer.

Application of Lubricated Granules

The lubricated granules of the invention may suitably be incorporated in compositions for use in industry or household. Such compositions include cleaning and/or disinfecting compositions such as detergent composition further comprising a surfactant. Such compositions are described e.g. in WO 00/01793 in the section “Detergent discosure”. Other compositions are antimicrobial compositions, wherein the enzyme is optionally an antimicrobial oxidoreductase and compositions for treatment of microbial biofilm, wherein the enzyme is optionally an antimicrobial oxidoreductase and the composition optionally further comprises a hydrolase enzyme. Other composition may be feed or food compositions such as animal feed or bakers flour.

### EXAMPLES

### Example 1

An uncoated Savinase (a protease) containing granulate was produced as described in U.S. Pat. No. 4,106,991 example 1 with the following exceptions:

Sodium sulfate was used in stead of sodium chloride as filler material

The enzyme concentrate was an aqueous suspension of crystalline enzyme containing also a carbohydrate binder (Glucidex) and methionine as an antioxidant.

The granulate was coated as described in U.S. Pat. No. 4,106,991 Example 22 by applying a solution of 6.9% PEG 4000 and 12.5% of a 1:1 TiO₂/Kaolin mixture. The granulate was further coated in a Lödige mixer with 2.0% PEG4000 followed by powdering the granulate with 1.5% Kaolin.

A part of the granulate was lubricated by adding a nonionic surfactant in a Lödige mixer (mixing for 5 minutes at room temperature) as given in the table below. The final granulates were tested for dust using the conventional Heubach attrition method which is known to the art, (see e.g. WO 93/07263), measuring both the total dust created (as mg dust per 20 g granulate) and the enzyme dust (as nanogram protein per g granulate). The values given in the table is the mean of three measurements.

| | | | Active |
|---|---|---|---|
| | | | Savinase dust |
| | | Total dust | in ng |
| Granulate | Lubricant | in mg/20 g | protein/g |
| 1.1 | None | 1.0 | 287 |
| 1.2 | 0.25% Softanol | 0.2 | 65 |
| | 50 | | |
| 1.3 | 0.50% Softanol | 0.3 | 32 |
| | 50 | | |
| 1.4 | 0.25% Dobanol | 0.2 | 39 |
| | 25-7 | | |
| 1.5 | 0.50% Dobanol | 0.2 | 7 |
| | 25-7 | | |

This example demonstrates that a final lubrication of the granulate with nonionic surfactant in an amount of 0,25% w/w or 0.50% w/w significantly reduces the dust levels.

### Example 2

Uncoated Enzyme Granulate was Produced as in Example 1

The granulate was coated as described in example 1 by applying a solution of 5.0% PEG 4000 and 12.5% of a 1:1 TiO₂/Kaolin mixture.

The granulate was further coated with 1.5% PEG4000 in a Lödige mixer, followed by powdering the granulate with 1.5% Kaolin.

After cooling a part of the granulate was lubricated by adding a mineral oil in a Lödige mixer (mixing for 5 minutes at room temperature) as given in the table below. The final granulates were tested for dust using the Heubach method. The values given in the table is the mean of three measurements.

| | | | Active |
|---|---|---|---|
| | | | Savinase dust |
| | | Total dust | in ng |
| Granulate | Lubricant | in mg/20 g | protein/g |
| 2.1 | none | 9.7 | 1209 |
| 2.2 | 0.50% Whiteway | 0.4 | 1.5 |
| | T15 | | |

This example demonstrates that a final lubrication of the granulate with mineral oil significantly reduces the dust levels.

### Example 3

Uncoated Enzyme Granulate was Produced as in Example 1

The granulate was coated as described in example 1 by applying a solution of 4.5% PEG 4000 and 12.5% of a 1:1 TiO₂/Kaolin mixture. The granulate was further coated in a Lödige mixer with a pre-blended mixture of PEG4000 and lubricant (see table below), followed by powdering the granulate with 1.5% Kaolin. Both a lubricant soluble in PEG4000 (Softanol 50) and a lubricant not soluble in PEG4000 (Whiteway T15) were used. The final granulates were tested for dust using the Heubach method. The values given in the table is the mean of three measurements.

| | | | | Active |
|---|---|---|---|---|
| | | | | Savinase dust |
| | | | Total dust | in ng |
| Granulate | PEG4000 | Lubricant | in mg/20 g | protein/g |
| 3.1 | 2.00% | none | 5.1 | 1363 |
| 3.2 | 1.50% | 0.50% Softanol | 4.3 | 951 |
| | | 50 | | |
| 3.3 | 1.50% | 0.50% Whiteway | 0.7 | 166 |
| | | T15 | | |

This example demonstrates, that if lubricant is mixed with the coating material (PEG4000) the best effect comes from a lubricant that is not soluble in the coating material.

### Example 4

The relative friction coefficient of the lubricated granulates from example 1 were measured using a powder rheometer from Manumit Powder Rheometer (Freeman Technology FT3 Powder Rheometer). The friction coefficient is proportional to the FORCE divided by the TORQUE measured by the rheometer (tip speed 50 rpm, helix angle 3° (compaction mode), using the 46 mm rotor and weighing 170 g granulate into the 50 mm container. The relative friction coefficients are calculated by dividing with the friction coefficient from the un-lubricated granulate:

(FORCE/TORQUE)_{SAMPLE}/(FORCE/TORQUE)_{REFERENCE}

Results

| Lubricant | Relative Friction Coefficient |
|---|---|
| None | 100% |
| 0.25% Softanol 50 | 78% |
| 0.5% Softanol 50 | 70% |
| 0.25% Dobanol 25-7 | 72% |
| 0.5% Dobanol 25-7 | 68% |

The example demonstrates that the relative friction coefficient is significantly lowered by lubrication

1. An active-containing granule comprising a lubricant on the outer surface of the granule, wherein:
(a) the lubricant is in liquid form at 25° C. and has a viscosity of less than 5000 centipoise at 25° C.; or
(b) the lubricated granule has a relative friction coefficient which is less than 80% when compared to an unlubricated granule when measured by a rheometer by using a tip speed of 50 rpm, a helix angle of 3° (compaction mode), using the 46 mm rotor and weighing 170 g granulate into a 50 mm testing container; or
(c) the lubricant is a mineral oil; or
(d) combinations thereof.

2. The granule of claim 1, wherein the active contained in the granule is a biologically active material.

3. The granule of claim 1, wherein the active contained in the granule is a pharmaceutically active material.

4. The granule of claim 1, wherein the active contained in the granule is a catalytically active material.

5. The granule of claim 1, wherein the active contained in the granule is a peptide or a polypetide.

6. The granule of claim 1, wherein the active contained in the granule is an enzyme.

7. The granule of claim 1, wherein the lubricant is less than 1% w/w of the granule.

8. The granule of claim 1, wherein the lubricant is less than 0.75% w/w of the granule.

9. The granule of claim 1, further comprising one or more coating layers.

10. The granule of claim 9, wherein the lubricant is insoluble in the coating layer on to which it is applied.

11. The granule of claim 1, wherein the lubricant of said (a) or said (b) is an organic compound or a mixture of organic compounds.

12. The granule of claim 11, wherein the organic compound or at least one organic compound in the mixture of organic compounds is a hydrophobic compound.

13. The granule of claim 1, wherein the lubricant is an animal oil.

14. The granule of claim 1, wherein the lubricant is a mineral oil.

15. The granule of claim 1, wherein the lubricant is a plant oil.

16. The granule of claim 1, wherein the lubricant is a non-ionic surfactant.

17. The active-containing granule of claim 1, wherein the lubricant is in liquid form at 25° C. and has a viscosity of less than 5000 centipoise at 25° C.

18. The active-containing granule of claim 1, wherein the lubricated granule has a relative friction coefficient which is less than 80% when compared to an unlubricated granule when measured by a rheometer by using a tip speed of 50 rpm, a helix angle of 3° (compaction mode), using the 46 mm rotor and weighing 170 g granulate into a 50 mm testing container.

19. A process for producing granules of claims 1-18, wherein an active-containing granule is contacted with a lubricant, so as to deposit lubricant on the outer surface of the granule.

20. A detergent composition comprising the lubricated active-containing granule of claims 1-18, and further comprising a surfactant.

21. A composition comprising the lubricated active-containing granule of claims 1-18.
